(19) United States
(12) Patent Application Publication
(10) Pub. No.: US 2018/0274005 A1
(43) Pub. Date: Sep. 27, 2018

(54) CORD BLOOD THERAPY TO TREAT CHRONIC DISEASE CAUSED BY L-FORM BACTERIA

(71) Applicant: Soft Cell Biological Research, LLC, St. George, UT (US)

(72) Inventor: John Brent Hunt, St. George, UT (US)

(21) Appl. No.: 15/989,132

(22) Filed: May 24, 2018

Related U.S. Application Data
(60) Provisional application No. 62/476,320, filed on Mar. 24, 2017.

(51) Int. Cl. C12Q 1/18; A61K 35/51

(52) U.S. Cl. CPC C12Q 1/18; A61K 35/51

(57) ABSTRACT

The present disclosure describes screening cord blood for antibacterial activity against L-form bacteria, methods of treating a patient having an L-form bacterial infection using a cord blood agent, and methods of killing or inhibiting L-form bacteria in an in vitro or ex vivo setting. L-form bacteria underlying an L-form bacterial infection are isolated from a subject having an infection and are altered to a culturable morphology. A set of cord blood agents are screened against the isolated L-form bacteria to identify one or more cord blood agents usable as effective treatment agents.

### RELATED APPLICATIONS

This Application claims the benefit under 35 U.S.C. § 119(e) of the filing date of U.S. Provisional Application Ser. No. 62/476,320, filed Mar. 24, 2017, the entire contents of which are incorporated herein by reference.

### BACKGROUND

Cord blood is the blood that remains within the umbilical cord and placenta following childbirth and cutting of the umbilical cord. Cord blood is known to include hematopoietic stem cells, and is often collected and banked for this reason. In medical use, cord blood is transplanted to patients with hematopoietic or genetic disorders. For example, in circumstances where bone marrow is not providing sufficient blood cell generation (a common condition following blood cancer radiation treatment), a cord blood infusion may be utilized to reconstitute the bone marrow. A cord blood infusion may also be administered to a patient suffering from anemia, in an attempt to induce greater production of healthy red blood cells.

L-form bacteria are bacteria lacking a full cell wall structure and are distinguished from typical morphologies of bacteria which exhibit cell walls. Standard culturing and detection methods are directed to bacteria exhibiting cell walls. These standard tests often fail to detect the presence of L-form bacteria within a sample, making them challenging to culture and characterize.

### SUMMARY

Aspects of the present disclosure relate to methods of screening one or more cord blood samples for antibacterial activity against L-form bacteria, using cord blood to treat a subject with an L-form bacterial infection, and/or using cord blood (e.g., whole cord blood, one or more fractions of cord blood, or components, such as molecules or cells, isolated from cord blood) to kill or deactivate L-form bacteria in an in vitro or ex vivo environment.

In some embodiments, one or more cord blood samples are contacted against an L-form bacteria of interest. Based on the presence or absence of an antibacterial effect against the L-form bacteria of interest (e.g., inhibition of growth of the L-form bacteria), the one or more cord blood samples are identified as effective against L-form bacteria. Cord blood samples may include whole cord blood and/or may include one or more cord blood fractions or components (e.g., molecules, cells etc.) isolated from cord blood or cord blood fractions.

The L-form bacteria of interest may be isolated from a subject infected by the L-form bacteria of interest. The subject may be treated with one or more cord blood agents (e.g., whole cord blood, one or more cord blood fractions, or one or more components isolated from cord blood, etc.) identified as being effective against the L-form bacteria of interest. For example, the subject may be treated with a blood transfusion to administer the one or more effective cord blood agents.

In some embodiments, a method of treating a subject having an L-form bacterial infection includes: (1) identifying a subject having an L-form bacterial infection, and (2) administering an effective dose of a cord blood agent (e.g., whole cord blood, one or more cord blood fractions or components (e.g., molecules, cells etc.) isolated from cord blood, etc.) to the subject. In some embodiments, the cord blood agent inhibits (e.g., kills or deactivates) L-form bacteria causing the L-form bacterial infection.

In some embodiments, a cord blood agent (e.g., whole cord blood, one or more cord blood fractions, etc.) is utilized to kill or inhibit growth of L-form bacteria in an in vitro or ex vivo environment. A method for killing or inhibiting L-form bacteria in an in vitro or ex vivo environment comprises: (1) optionally, providing a culturable form of an L-form bacteria in vitro or ex vivo, and (2) contacting the L-form bacteria to a cord blood agent.

In some aspects, the disclosure relates to use of a cord blood agent (e.g., whole cord blood, one or more cord blood fractions or components (e.g., molecules, cells etc.) isolated from cord blood, etc.) in treating a subject having an L-form bacterial infection. In some embodiments, the use comprises administering to the subject a cord blood agent. In some embodiments, the use comprises screening a sample obtained from the subject for the presence of L-form bacteria (e.g., using a method as described by the disclosure), and optionally, screening one or more cord blood agents for activity against the L-form bacteria, prior to the administering. In some embodiments, the cord blood agent is selected based on the results of the screening (e.g., a cord blood agent shown to inhibit L-form bacteria in the sample is subsequently administered to the subject).

In some aspects, the disclosure relates to the use of a cord blood agent for inhibiting L-form bacteria in vitro or ex vivo. In some embodiments, the use comprises contacting an L-form bacteria with a cord blood agent that has previously been identified as having activity against (e.g., inhibiting or killing) L-form bacteria. In some embodiments, a cord blood agent is identified as having activity against L-form bacteria by a screening method as described by the disclosure.

Additional features and advantages will be set forth in part in the description that follows, and in part will be obvious from the description, or may be learned by practice of the embodiments disclosed herein. The objects and advantages of the embodiments disclosed herein will be realized and attained by means of the elements and combinations particularly pointed out in the appended claims. It is to be understood that both the foregoing brief summary and the following detailed description are exemplary and explanatory only and are not restrictive of the embodiments disclosed herein or as claimed.

### BRIEF DESCRIPTION OF THE DRAWINGS

In order to describe various features and concepts of the present disclosure, a more particular description of certain subject matter will be rendered by reference to specific embodiments which are illustrated in the appended drawings. Understanding that these figures depict just some example embodiments and are not to be considered to be limiting in scope, various embodiments will be described and explained with additional specificity and detail through the use of the accompanying drawings in which:

FIG. 1 illustrates an exemplary method for screening a sample for the presence of L-form bacteria;

FIG. 2 illustrates progression of an aging cell infected with L-form bacteria;

FIG. 3 illustrates a method for screening for L-form bacteria including comminution of the sample;

FIG. 4 illustrates an exemplary method for transferring an L-form inoculant from a liquid growth medium to a solid growth medium; and

FIG. 5 is a photograph of plated bacterial cultures treated with cord blood.

FIG. 6 is a photograph of plated bacterial cultures treated with cord blood.

FIG. 7 is a photograph of plated bacterial cultures treated with cord blood.

### DETAILED DESCRIPTION

L-form bacteria have been found to reside in the blood and other tissues of some individuals, including individuals otherwise appearing healthy and not otherwise showing symptoms of infection. In some circumstances, such L-form bacteria underlying an L-form infection may transition from a latent/asymptomatic form to a symptomatic form. In some circumstances, individuals having certain conditions (such as anemia, implant rejection, joint infection, etc.) may have an underlying L-form bacterial infection causing the symptoms and complications. These underlying infections may go undiagnosed because standard laboratory testing procedures fail to identify L-form bacteria and fail to diagnose the subject as having an infection.

Detecting the presence of such L-form bacteria within a subject's blood, and thereby diagnosing the subject as having an L-form bacterial infection, is not possible using conventional infection screening and diagnosing techniques. Further, conventional laboratory techniques are not able to culture the L-form bacteria within the subject's blood sample to a form where potential treatment agents can be tested against the L-form bacteria in an in vitro or ex vivo setting.

In contrast, methods are described herein which enable the isolation and culturing of L-form bacteria from a subject's blood sample. The methods provide for the diagnosis of a subject as having an L-form bacterial infection. Further, where an L-form bacterial infection is determined, the methods described herein further provide for the isolation, culturing, and identification of the L-form bacteria underlying the L-form bacterial infection.

Surprisingly, it has now been found that at least some L-form bacteria are susceptible to treatment using a cord blood agent (e.g., whole cord blood and/or cord blood fractions), which has been shown to often kill or inhibit the growth of cultured L-form capable bacteria. With the availability of culturable forms of L-form capable bacteria made possible using the methods described herein, potential antibacterial agents, including cord blood agents, may be tested against the isolated L-form bacteria to determine potential effectiveness.

The disclosure relates, in part, to cord blood samples that are effective against L-form bacteria (e.g., inhibit growth of, or kill, certain L-form bacteria), in some cases as a result of containing effective amounts of hematopoietic stem cells, transmembrane proteins, antimicrobial compounds, or combinations thereof.

Some embodiments of methods or uses described herein are directed to methods of screening cord blood for antibacterial activity against L-form bacteria. Some embodiments described herein are directed to methods of treating a patient having an L-form bacterial infection using cord blood. Some embodiments described herein are directed to killing or inhibiting L-form bacteria in an in vitro or ex vivo setting.

Accordingly, in some embodiments, methods described by the disclosure comprise screening for activity of cord blood samples (e.g., whole cord blood, one or more cord blood fractions or components (e.g., molecules, cells etc.) isolated from cord blood, etc.) against L-form bacteria. For example, an L-form bacteria culture may be contacted with a cord blood agent, and if growth of the L-form bacteria is inhibited by the cord blood agent, the cord blood agent is identified as having activity against that particular L-form bacteria.

### Definitions

As used throughout this disclosure, the terms “cell-wall-sufficient bacteria” (“CWS bacteria”) or “classic-form bacteria” refer to strains of bacteria having a morphology with an identifiable and recognizable cell wall structure, such as the thick peptidoglycan layer of Gram positive bacteria and the thin peptidoglycan layer positioned between the cell membrane and the outer membrane (lipopolysaccharide layer) of Gram negative bacteria. As used herein, the term CWS bacteria also refers to mycobacteria, bacteria within the archaea domain, and other forms of bacteria known to those of skill in the art to typically exhibit a cell wall structure, even if not necessarily easily categorized as Gram positive or Gram negative bacteria.

The terms “L-form bacteria,” “pleomorphic bacteria,” “hidden bacteria,” “intracellular bacteria,” “fastidious bacteria,” and the like do not have standard definitions. The terms are often used synonymously, but in some instances, for example, the term “intracellular bacteria” may refer to bacteria residing within a host cell regardless of level of cell wall formation of the bacteria.

As used herein, the term “L-form bacteria” refers to strains of bacteria often found to reside intracellularly within a host cell and which do not exhibit a full cell wall structure. Such bacteria are distinguished from typical cell-wall-sufficient bacteria for which traditional culturing and detection methods are directed. “L-form bacteria” include bacterial strains with morphologies lacking any identifiable cell wall structure or cell wall components, and include strains including an undeveloped or incomplete cell wall structure, such as strains containing some cell wall components but lacking sufficient structure to fully define the cell wall (e.g., strains with variable shape as opposed to typical cocci, rod, and/or spiral characterization). The skilled artisan will recognize that the term “L-form bacteria” does not, however, refer to bacteria of the genus Mycoplasma.

In some instances, the term “L-form bacteria” includes strains of bacteria that do not yet include fully recognizable cell wall structures, but which are transitioning toward cell wall sufficient strains. The term “L-form bacteria” also refers to pleomorphic bacteria which are capable of progressing from a reduced-cell-wall or absent-cell-wall-form toward a classic form with a full cell wall. The term also includes species and/or strains of bacteria that are not known to exist in nature in a CWS form, but which have been found to reside in one or more samples in L-form.

The term “L-form capable bacteria” is used herein to describe bacteria that are found within an L-form sample and which have been cultured from the L-form morphology into a CWS morphology. Such strains often exhibit flexible morphological characteristics and are able to revert back to an L-form morphology under certain environmental conditions.

Although the exemplary embodiments described herein refer specifically to bacteria, one of skill in the art will understand that certain principles disclosed herein may be utilized for culturing, screening, and/or detecting fungi (e.g., yeast), protozoans, and other pathogenic microorganisms capable of residing intracellularly within host cells and/or capable of being hidden from immune system responses within biological fluids or tissues.

As used herein, the term “sample” refers to a biological sample such as a tissue sample, whole blood sample, serum sample, plasma sample, and the like. Such samples are typically obtained from mammalian sources. As used herein, “sample” may also refer to mixtures containing the tissue/clinical sample. For example, a sample may be added to or mixed with a growth medium to promote the growth of bacteria within the sample. When such a mixture is further processed (e.g., transferred, analyzed, monitored, stored, etc.), the mixture may be referred to simply as the “sample.”

As used herein, “cord blood agent” refers to umbilical cord blood and/or one of its components from a human or other mammalian source. A cord blood agent may include whole cord blood and/or one or more fractions of whole cord blood. In some embodiments, a cord blood agent comprises one or more components, such as molecules, cells, etc., that are isolated or extracted from a cord blood agent. In some embodiments, a cord blood agent is a fraction or a sub-fraction of whole cord blood (e.g., a blood plasma fraction, a plasma protein fraction, blood serum fraction, etc.). In some embodiments, a cord blood agent is purified or sterilized, for example by filtration, chemical treatment, ultraviolet (UV) treatment, or any combination thereof.

### Screening Cord Blood for Activity Against L-Form Bacteria

In some embodiments, a method of screening one or more cord blood samples for antibacterial activity against L-form bacteria comprises: (1) optionally, providing an L-form bacteria of interest, (2) providing one or more cord blood samples, (3) contacting the one or more cord blood samples with an L-form bacteria of interest (e.g., that is present in or isolated from a biological sample), and (4) based on the presence or absence of an antibacterial effect against the L-form bacteria of interest (e.g., inhibition of growth or killing of L-form bacteria), identifying the one or more cord blood samples as effective against L-form bacteria.

The L-form bacteria of interest may be an L-form bacteria isolated from a subject infected with that particular strain of L-form bacteria. The screening may thereby provide treatment information relevant to treating the subject's L-form bacterial infection. In some embodiments, the disclosure relates to a method comprising (i) identifying a subject as having an L-form bacterial infection; (ii) screening a cord blood agent against a biological sample obtained from the subject; and, optionally (iii) recommending administration of the cord blood agent to the subject and/or administering the cord blood agent to the subject if the cord blood agent inhibits L-form bacteria in the biological sample.

In some embodiments, one or more cord blood samples are divided into blood fractions, and one or more of the particular blood fractions are contacted with the L-form bacteria of interest. For example, a cord blood sample may be fractionated into plasma, buffy coat, and erythrocyte fractions, with one or more of the separate fractions being tested against the L-form bacteria of interest. The foregoing fractions may be further fractionated to additional sub-fractions, with one or more of the sub-fractions being separately tested against the L-form bacteria of interest. For example, the blood plasma fraction may be further fractionated into various plasma protein fractions, with one or more of the plasma protein fractions being separately tested against the L-form bacteria of interest. Accordingly, in some embodiments, a cord blood fraction or sub-fraction (e.g., a blood plasma fraction, a plasma protein fraction, blood serum fraction, etc.) may be used as a cord blood agent.

In some embodiments, a cord blood sample may comprise one or more components that have been isolated or extracted from whole cord blood or a fraction of whole cord blood, for example small molecules (e.g., antibiotic molecules), nucleic acids (e.g., DNA, RNA, etc.), peptides, proteins, polypeptides (e.g., enzymes, antibodies, etc.), and/or cells (e.g., certain cell types, for example immune cells).

### Treatment of a Patient Using a Cord Blood Agent

In some embodiments, a method of treating a subject having an L-form bacterial infection comprises: (1) identifying a subject having an L-form bacterial infection, (2) administering an effective dose of a cord blood agent to the subject, and (3) the cord blood agent killing or deactivating L-form bacteria causing the L-form bacterial infection. The cord blood agent may be whole cord blood, or may be one or more fractions of cord blood.

The method of treating a subject may be utilized in conjunction with a method of screening for cord blood with activity against L-form bacteria as described above. For example, upon determining that one or more cord blood samples provide an antibacterial effect against the L-form bacteria of interest, the one or more cord blood samples may be utilized as the cord blood agent to treat a subject with an infection associated with the L-form bacteria of interest.

In one example, the subject having an infection of the L-form bacteria of interest may be treated through a cord blood transfusion. One or more cord blood agents which have shown an antibacterial effect against the L-form bacteria of interest may be utilized and/or may indicate a source of cord blood which may be utilized to treat the subject. (e.g., after appropriately blood typing to ensure compatibility). In some embodiments, one or more components (e.g., molecules or cells) are isolated or extracted from a cord blood sample that has shown an antibacterial effect against the L-form bacteria. In some embodiments, the one or more components are used to treat a subject having an infection of L-form bacteria.

The methods described herein can beneficially enable targeted cord blood therapy capable of treating an L-form bacterial infection. Without isolating and culturing the L-form bacteria underlying an L-form infection, and without screening of cord blood or cord blood fractions against the isolated L-form bacteria, any cord blood based therapy would essentially be random, with much lower chance of providing effective outcomes. In contrast, the methods described herein enable the isolation and culturing of L-form bacteria causing an L-form bacterial infection, the screening of cord blood agents against the isolated and cultured L-form bacteria in order to identify one or more effective cord blood agents, and the use of the one or more cord blood agents to treat the L-form bacterial infection.

### Using a Cord Blood Agent Against L-Form Bacteria In Vitro or Ex Vivo

In some embodiments, a cord blood agent (whole cord blood and/or one or more cord blood fractions) is utilized to kill or inhibit L-form bacteria in an in vitro or ex vivo environment. A method for killing or inhibiting L-form bacteria in an in vitro or ex vivo environment comprises: (1) providing a culturable form of an L-form bacteria in vitro or ex vivo, (2) contacting the L-form bacteria to a cord blood agent, and (3) the cord blood agent killing or deactivating the L-form bacteria.

For example, a cord blood agent may be applied to an L-form bacterial culture growing on solid media (e.g., plates, slants) or to an L-form bacterial culture growing in liquid media. In some embodiments, a cord blood agent may be applied to L-form bacteria within a biological sample, such as an extracted blood sample, synovial fluid sample, biopsy, or other biological sample capable of harboring L-form bacteria.

### Methods of Screening for L-Form Bacteria

Methods of culturing L-form bacteria are generally described, for example by U.S. Publication No. 2016/0168614, the entire contents of which are incorporated herein by reference.

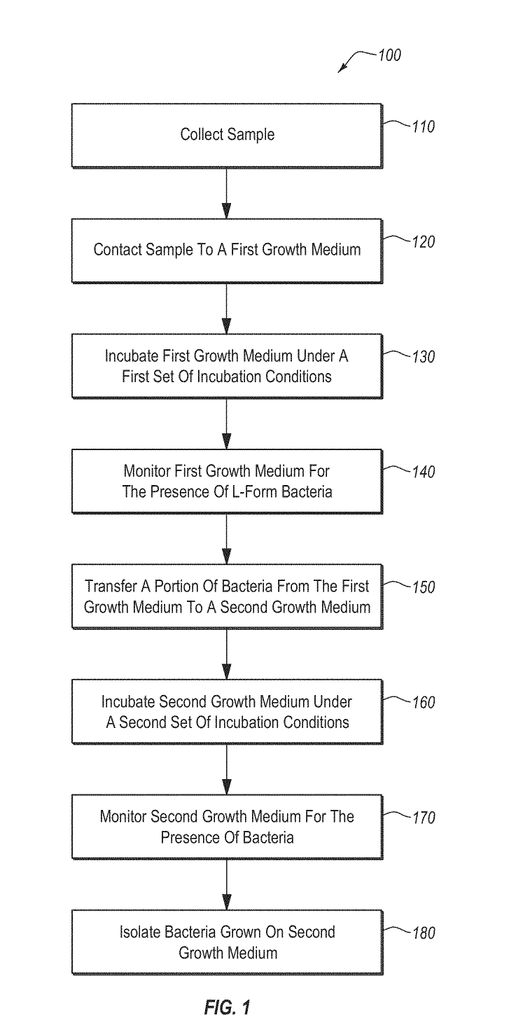

FIG. 1 illustrates an exemplary method 100 of screening a sample for the presence of L-form bacteria and culturing/producing L-form capable bacteria. In some embodiments, the method includes a step 110 of collecting a sample, a step 120 of contacting the sample to a first growth medium, a step 130 of incubating the inoculated first growth medium under a first set of incubation conditions, and a step 140 of monitoring the inoculated first growth medium for the presence of L-form bacteria.

In some embodiments, the step 110 of collecting the sample is performed using standard sample collection techniques, such as a blood draw, tissue swab, and the like. In some embodiments, the sample is collected in the same container in which the first growth medium is contained. Alternatively, the sample may be collected in one or more separate containers prior to storage, transport, and subsequent transfer to the container holding the first growth medium.

Various types and/or combinations of growth media may be used as the first growth medium. For example, the first growth medium may be formulated as complex growth media (e.g., blood, yeast extract, bile, peptone, serum, and/or starch containing medias), defined growth media, or a selective media (e.g., nutrient selective for mannitol, cysteine, lactose, sucrose, salicin, xylose, lysine, or combinations thereof; selective based on carbon source, nitrogen source, energy source, and/or essential amino acids, lipids, vitamins, minerals, trace elements, or other nutrients; and/or selective antibiotic/antimicrobial containing media). Exemplary growth media that may be used in solid (e.g., with agarose) or liquid form include R2A, nutrient, chocolate blood, blood, mannitol salt, Vogel Johnson, Kligler iron, Simmons citrate, Columbia, cetrimide, xylose-lysine-deoxycholate, tryptic soy, Tinsdale, Phenylethyl alcohol, Mueller-Hinton, MacConkey, brain-heart infusion (BHI), and lysogeny broth media.

In preferred embodiments, the first growth medium is a liquid growth medium. In one particular example, the growth media is serum (e.g., human, bovine) and/or brain-heart infusion (BHI) broth, and may be contacted with the sample as a liquid in suspension with the sample. In preferred embodiments the growth media is formulated without substances that would hamper or restrict the growth of any bacteria found within the sample. For example, the growth media preferably omits antimicrobial enzymes (e.g., lysozyme, protease, etc.), antimicrobial peptides, and immune system components (e.g., leukocytes, complement system proteins, antibodies or other immunoglobulins, etc.).

For example, it has been discovered that L-form bacteria are often able to reside within a sample at a low-grade level without eliciting a full immune response and without converting to classic form. The presence of immune system components or other growth hampering substances within such samples can prevent the bacteria from being manifest in classic form, even though the bacteria are present within the sample in L-form. Under such circumstances, the removal or dilution of growth hampering substances and/or the transfer of L-form bacteria to growth media without growth hampering substances can promote progression of the bacteria within the sample to classic form, and thereby provide faster culture and screening of L-form bacteria within the sample.

In some embodiments, the first set of incubation conditions promote the aging of the sample tissue cells, allowing L-form bacteria present within the cells to grow. For example, as the cells die and rupture, more L-form bacteria are able to escape their intracellular positions and move into the surrounding extracellular medium. In addition, the dilution of the sample within the first growth medium dilutes the concentration of antibodies and other immune system components present within the blood sample, also enabling greater growth of the L-form bacteria.

In some embodiments, immune system components may be removed from the sample or from the inoculated first growth medium, or can be inactivated by adding an inactivating agent, such as a binding compound or complement inactivator, by adding one or more blocking antibodies, by washing, centrifuging, and/or filtering the sample to separate cells from other immune system molecules, or simply by diluting the sample sufficiently within the growth medium to render the components ineffective. In preferred embodiments, however, substances that would hamper polymerase chain reaction (PCR) or other analysis techniques (such as ethylenediaminetetraacetate (EDTA)), or that would inhibit conversion/reversion to classic form (such as EDTA), are omitted.

After the sample is contacted with the first growth medium in step 120, the method proceeds to step 130 by incubating the inoculated first growth medium under a first set of incubation conditions. The collected sample is stored at a temperature about body temperature or at a temperature lower than about body temperature. For example, the collected sample may be stored at a temperature, constant or fluctuating, within a range or about 20° C. to about 40° C., or within a range of about 25° C. to about 35° C., or more preferably within a range of about 25° C. to about 30° C., or about 27° C. In preferred embodiments, the inoculated first growth medium is stored at a temperature below body temperature.

It has been surprisingly found that L-form bacteria within a sample grow at a greater rate at temperatures lower than body temperature. For example, in human blood samples, which are typically stored at body temperature (37 degrees Celsius), it has been found that storage at a lower temperature increases the growth of L-form bacteria within the sample and enables L-form bacteria which would otherwise remain present at non-detectable levels to grow to observable levels. Preferably, incubation also omits rocking or shaking of the growth medium in order to reduce the amount of contact between any L-form bacteria and any antibodies or other immune components that may be present within the sample.

The inoculated first growth medium is incubated for a time sufficient to provide growth of any L-form bacteria present within the sample (e.g., for a time sufficient to allow any L-form bacteria present within the sample to achieve a detectable population). In some embodiments, this monitoring period can be about 120 hours or even longer than 120 hours. In more preferred embodiments, this monitoring period can be less than about 120 hours. For example, in some embodiments, the monitoring period can be within a range of about 24 hours to about 96 hours, or within a range of about 36 hours to about 84 hours. In other embodiments, the monitoring period is within a range of about 48 hours to about 72 hours.

In some embodiments, a dual track culturing setup is followed by subjecting a first set of sample portions to a short-track monitoring period and a second set of sample portions to a long-track monitoring period, where the short and long-track monitoring periods have durations according to the above ranges, with the short-track duration being shorter than the long-track duration. Such a dual-track setup has shown good results by enabling faster results from the short-track, when possible, without missing the detection of other, slower species and/or strains resulting from the long-track.

Step 140 of monitoring the first growth medium during the monitoring period for the presence of any L-form bacteria may be performed by transferring the sample or a portion of the stored sample to a microscope slide, well plate, or other such apparatus allowing the microscopic visualization of the sample or portion of the sample. In preferred embodiments, in order to avoid the disruption of potentially fragile L-form bacteria within the sample or portion of the sample collected for microscopic inspection, the visual monitoring is carried out without traditional staining (e.g., Gram staining) or chemical or heat fixing steps. For example, the visual monitoring may be carried out by direct microscopic observation of the sample or portion thereof by preparing a wet-mount, live slide for observation. Although microscopy using live slides is the preferred manner of monitoring for L-form growth, other suitable monitoring techniques include spectrophotometric methods (including colorimetry and measurement of optical density), staining, and measurements of turbidity, total cellular DNA and/or protein levels, electrical field impedance, bioluminescence, carbon dioxide, oxygen, ATP production or consumption, and the like.

Monitoring of the first growth medium may be carried out throughout the monitoring period. For example, monitoring may occur periodically according to a set schedule throughout the monitoring period, such as at set intervals (e.g., daily, every 12 hours, every 10, 8, or 6 hours, every 4, 3, or 2 hours, hourly, or even more frequently). In some circumstances, a sample may be monitored throughout a monitoring period, and may fail to exhibit any indication of bacterial presence. At this point, in some embodiments, the method is completed and a negative result is returned (e.g., the method either detected or failed to detect the presence of any L-form bacteria in the sample).

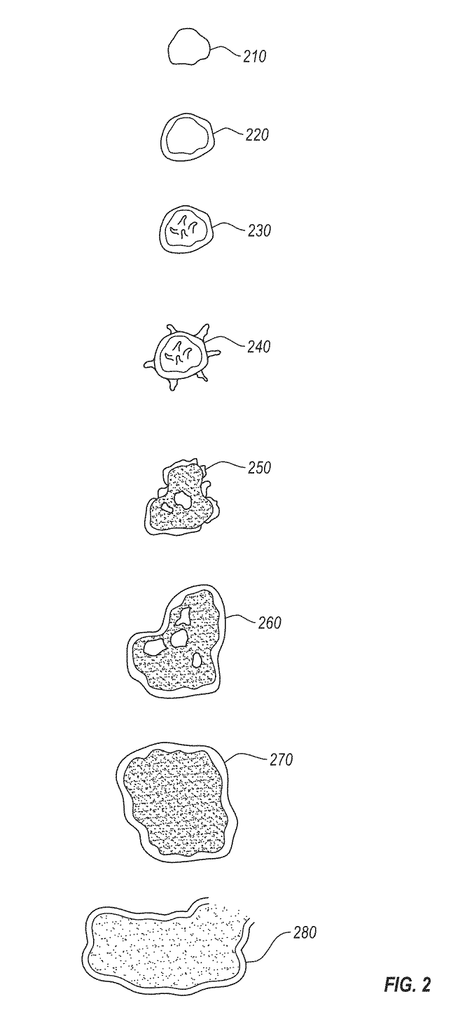

Prior to transferring to a second growth medium, the inoculated first growth medium is preferably incubated until L-form bacteria within the medium have progressed to a state of sufficient growth. FIG. 2 illustrates a typical progression of a red blood cell harboring L-form bacteria once placed under the first set of incubation conditions. A healthy red blood cell 210 that harbors L-form bacteria will begin to progress to a first state 220, where internal pressure is created by developing L-form bacteria within the cell. At a second state 230, L-form bacteria begin to transition from a non-microscopically observable form (e.g., under about 0.05 μm) to an observable form. At a third state 240, internal structures of the red blood cell begin to break down (e.g., through the action of lysozymes), freeing up additional nutrients for L-form growth and creating greater internal pressure within the cell. In some circumstances it has been observed that many cells stay at this state for long periods of time (e.g., several weeks or months). L-form bacteria appear to be present in such cells, but the L-form bacteria are not released from the cells at detectable levels. When these types of cells are present, embodiments utilizing a comminuting step may be particularly advantageous.

In other circumstances, cells continue toward more progressed states. At a fourth state 250, outward protrusions of the cell become visible through weak spots in the wall of the degrading red blood cell. At a fifth state 260 and a sixth state 270, the cell wall further breaks down and the cell continues to expand toward its limits. At a seventh state 280, the cell ruptures due to degradation and excessive internal bacterial growth, releasing L-form bacteria into the surrounding growth medium.

Preferably, the inoculated first growth medium is incubated until at least some (e.g., 10% or more, 25% or more, 50% or more, 75% or more, 90% or more) of the monitored cells of the sample have progressed to a state where they have ruptured to release intracellular L-form bacteria.

Some embodiments further include a step 150 of transferring at least a portion of the inoculated first growth medium to a second growth medium, and a step 160 of incubating the second growth medium under a second set of incubation conditions. In preferred embodiments, the second growth medium is a solid-phase growth medium (e.g., contained in a plate or slant). For example, solid-phase growth media may include one or more of the growth media described above (e.g., complex media, defined media, minimal or selective media) incorporated into a solid substrate. Suitable solid substrates include those formed with agarose, collagen, laminin, elastin, peptidoglycan, fibronectin, and the like.

The second set of incubation conditions includes a temperature within a range of about 20° C. to about 40° C. Preferably, the second growth medium is incubated at approximately body temperature (about 30° C. to 40° C. or about 37° C.). The second growth medium is incubated at this temperature for a time period of about 24 hours to 96 hours, or about 36 hours to 84 hours, or about 48 hours to 72 hours, or about 60 hours. In some embodiments, the temperature is then adjusted to a range that is below body temperature (e.g., about 25° C. to 35° C., or about 25° C. to 30° C., or about 27° C.) for a time period of about 4 days to about 30 days, or about 7 days to about 21 days, or about 14 days. In preferred embodiments, the temperature is adjusted to a range that is below body temperature for a time period of about 1-7 days, or about 3-5 days.

In some embodiments, the step 150 includes transfer to multiple types of solid-phase growth media in order to isolate multiple strains that may be present within the sample. For example, a set of agar plates may be prepared to receive the sample, with several of the agar plates containing different forms of media (such as any of those types discussed above with respect to the sample collection device, including selective growth media), and these may be further divided by placing one set under aerobic conditions after inoculation and another set under anaerobic conditions after inoculation (e.g., by placing in a standard anaerobic chamber maintained with carbon dioxide). During or after incubation, the method can include the step 170 of monitoring the second growth medium for bacterial growth (e.g., using one or more of the monitoring techniques described herein).

Although defined medias may be used as growth media in the methods described herein, it has been found that L-form bacteria are able to be efficiently cultured and detected using various complex medias such as BHI medias or those including serum (as the first and/or second growth medias). Beneficially, the methods described herein have enabled the screening of L-form bacteria without the need for generally more expensive defined medium formulations. Without being bound to any particular theory, it is thought that one or more process steps, such as the particular incubation conditions (e.g., time, temperature) and/or transfer steps (e.g., transferring bacteria in a manner that enables bacteria within a sample to maintain a hydrated state) enables L-form bacteria to be cultured without the need for custom-made or defined medias.

Referring back to FIG. 1, some embodiments further include a step 180 of isolating bacteria grown on the second growth medium. As growth occurs on the second growth medium, some strains of L-form bacteria may transition to classic form morphologies and may grow classic form colonies on the second growth medium. Such bacteria may be transferred to separate media (e.g., one or more complex, selective, or defined medias described herein) until a single strain is found on the media, and/or may be sampled and further analyzed according to well-known microbiological characterization techniques, including microscopic examination, staining (e.g., Gram, Malachite green/Safranin, and acid-fast stains), and selective growth testing. Other analytical techniques such as chromatography, gel separation, immunoassays, flow-through assays (e.g., plasmon resonance detection), fluorescent probe binding and measurement, automated cell/plate counting, microwell reading, DNA hybridization and amplification methods (e.g., polymerase chain reaction, strand displacement amplification), 16S rDNA sequencing, other molecular biological characterization techniques, and combinations thereof may also be used to analyze bacteria cultured or isolated using the methods described herein.

Beneficially, many of the bacteria cultured to a CWS form using one of the culturing embodiments described herein maintain a flexible morphology capable of reverting back to L-form when exposed to appropriate conditions.

Any of the foregoing embodiments may also include the addition of a transmembrane protein inhibiting agent to promote and/or augment the conversion of L-form bacteria to CWS form. It is presently theorized that at least some L-form bacteria are capable of resisting a host immune response by modulating or secreting one or more of the host's transmembrane proteins in order to inhibit or dampen the host's immune response. In one example, an L-form bacteria may modulate or secrete the CD47 protein in order to inhibit macrophage response as a result of the CD47 protein engaging with SIRP-α. It is presently believed that inhibiting one or more of such transmembrane proteins in a collected sample (e.g., blood sample) will trigger or augment the conversion of L-form bacteria to CWS form. Some embodiments may therefore include the removal of one or more transmembrane proteins and/or the addition of an inhibiting agent (e.g., a targeted antibody) to inhibit one or more transmembrane proteins in order to trigger or augment the conversion of L-form bacteria to CWS form.

### Sample Comminution

In some circumstances, it may be desirable to subject a sample to blending, vortexing, sonication, or other disruptive processes or combinations thereof in order to disassociate biofilms and/or aggregates, to rupture cells, or to otherwise disperse any bacteria and increase exposure to surrounding growth media prior to further screening. It has been surprisingly found that proper use of a comminution step in a screening process can increase yields, reduce culture times, and allow for faster detection and screening of samples having L-form bacteria. Although the exemplary method may be used to prepare any of the forms of samples defined above, it may be particularly useful in preparing samples known to contain, or known to be likely to contain, biofilms, root nodules, and/or other aggregates potentially harboring L-form growth.

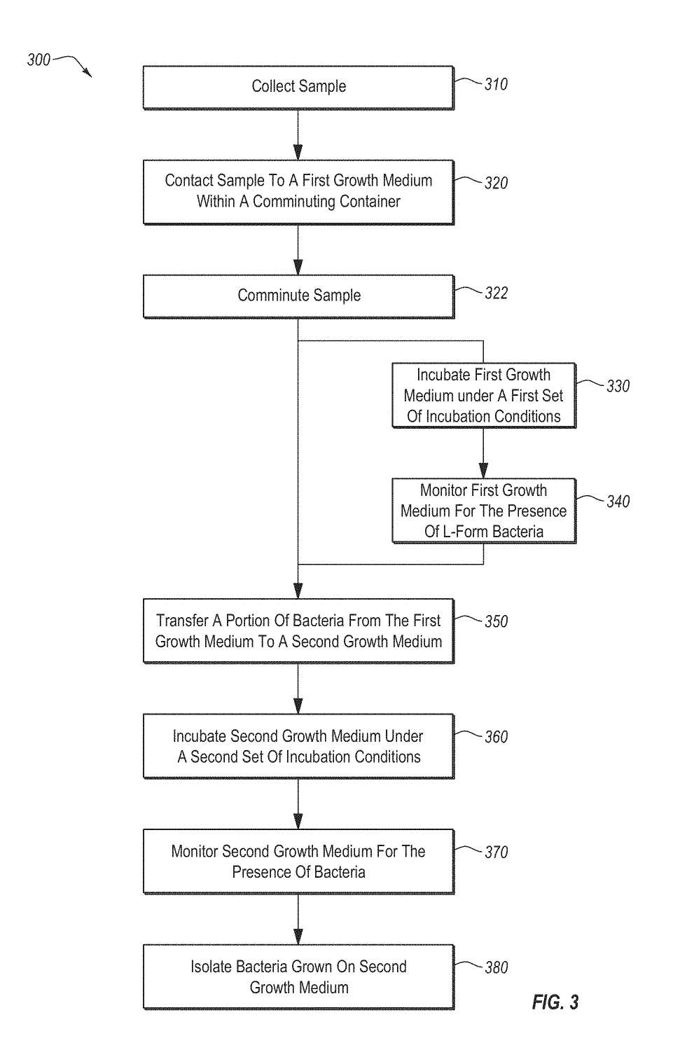

FIG. 3 illustrates another exemplary method 300 of screening for L-form bacteria that includes comminution of the sample. The embodiment shown in FIG. 3 has steps and elements similar to the embodiment shown in FIG. 1, and like numbers represent like elements. As illustrated, the method includes a step 310 of collecting a sample, and a step 320 of contacting the sample to a first growth medium. In some embodiments, the first growth medium is contained within a comminution container. The comminution container is typically formed as an elongate tube with a rounded bottom portion, or with a tapering (e.g., conical frustum) shaped bottom portion.

The comminution container includes a comminuting media configured to contact the sample and disaggregate biofilms, cell clumps, and other aggregates within the sample. The comminuting media is preferably formed from crushed or shattered glass. Other embodiments may include comminuting media formed from beads, shards, particles, fragments, filaments, or other structures configured to contact the sample and disassociate particles within the sample, and may be formed out of metal, plastic, ceramic, or other materials or combinations of materials.

The exemplary method includes a step 322 of comminuting the sample. In some embodiments, the sample and first growth medium are vortexed (e.g., by placing the comminuting container in a vortex apparatus) to displace the comminuting media within the liquid and to enable contact between the comminuting media and the aggregated portions of the sample. In other embodiments, the sample may be comminuted using magnetic stirring (e.g., one or more magnetic stir bars included in the comminuting media), or by shaking, vibrating, or otherwise displacing the comminuting media.

In some embodiments, after comminuting, the method includes a step 330 of incubating the inoculated first growth medium under a first set of incubation conditions and a step 340 of monitoring the inoculated first growth medium for the presence of L-form bacteria. Alternatively, after comminuting, the method can proceed to a step 350 of transferring a portion of the first growth medium to a second growth medium (preferably a solid growth medium) without prior incubation of the sample. Such embodiments can beneficially reduce the culture time required before bacteria can be isolated, analyzed, and/or harvested. For example, the progression of infected red blood cells shown in FIG. 2 can be effectively bypassed or made to progress more rapidly.

In some embodiments, the method then proceeds through a step 360 of incubating the second growth medium under a second set of incubation conditions, a step 370 of monitoring the second growth medium for the presence of bacteria, and optionally a step 380 of isolating bacteria grown on the second growth medium, as described above.

### Inoculant Transfer

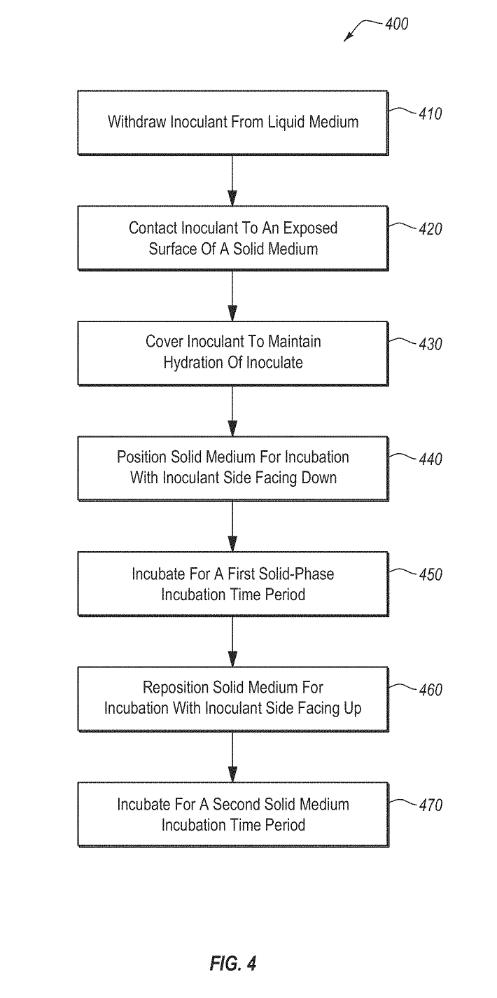

FIG. 4 illustrates an exemplary method 400 for transferring an inoculant from a first, liquid growth medium to a second, solid growth medium and incubating the solid growth medium (e.g., as part of the steps 150 and 160 in the embodiment of FIG. 1 or the steps 350 and 360 in the embodiment of FIG. 3). As shown, the method includes a step 410 of withdrawing an inoculant from the liquid medium, and a step 420 of contacting the inoculant to a surface of a solid medium.

After contacting the inoculant to the solid medium, the method includes a step 430 wherein the inoculant is immediately (e.g., within seconds or within about 1 or 2 minutes) covered by an insert in order to maintain a hydrated state of the inoculant. It has been found that positioning the insert over the inoculant beneficially enables L-form bacteria within the inoculant to interface with the solid substrate to begin colonization of the solid medium. It is theorized that L-form bacteria are often in a hydraulically fragile state at this point in culturing (e.g., due to reduced or absent cell wall structures), and that excessive drying and/or too rapid concentrating of solutes within the inoculant containing the L-form bacteria can inhibit further culturing of the L-form bacteria.

In some embodiments, the insert is a glass panel, glass slide, or other material configured to sit upon the solid media and preferably, to maintain position relative to the solid media (e.g., through adhesive forces between the inner surface of the insert contacting the inoculant and the inoculant). Other embodiments may include inserts made from rigid or film plastics, ceramics, or other materials. Preferably, the insert is positioned to eliminate air pockets within the inoculant between the surface of the solid media and the inner surface of the insert. In some embodiments, an additional amount of inoculant may be contacted to other portions of the surface of the solid media not covered by the insert, if any.

In some embodiments, the method further includes a step 440 of positioning the solid medium for incubation with the inoculant side facing down. For example, where an agarose plate is used to contain the solid media, the plate is positioned “upside down” so that the surface to which the inoculant and insert were applied faces down.

In some embodiments, the method further includes a step 450 of incubating the solid medium for a first solid-phase incubation time period of about 4 hours to 24 hours, or about 6 hours to 18 hours, or about 12 hours. The incubation may be carried out under the temperature conditions described in relation to step 160 of FIG. 1. Preferably, the incubation is also carried out in an atmosphere having a relative humidity that is sufficient to prevent overly rapid drying of the inoculant.

As explained above, it has been discovered that greater culturing efficiency is made possible by maintaining a hydrated state of the inoculants and growth media as the disclosed methods are performed. For example, during the first solid-phase incubation time period, the relative humidity may be maintained within a range of about 40% to about 100%, or about 50% to about 90%, or about 60% to about 80%. In some embodiments, the method further includes a step 460 of repositioning the solid medium with the inoculant side up. It has been discovered that, at this point in the progression of L-form cultures, the L-form bacteria have typically progressed enough and/or the insert has sufficiently interfaced with the solid medium, such that the benefits of repositioning the solid medium to allow evaporation of water that has built up in the inverted position outweigh the detrimental effects, if any, of repositioning.

In some embodiments, the method further includes a step 470 of incubating the solid medium for a second solid-phase incubation period. The second solid-phase incubation time period is preferably performed in an atmosphere having similar relative humidity levels of the first solid-phase incubation time period, and for a time period ranging from about 12 hours to about 84 hours, or about 24 hours to about 72 hours, or about 36 hours to about 60 hours, or about 48 hours. In some embodiments, one or more cultures are further incubated at a temperature in a range that is below body temperature (e.g., about 25° C. to about 35° C., or about 25° C. to about 30° C., or about 27° C.) for a time period of about 4 to about 30 days, or about 7 to 21 about days, or about 14 days. In preferred embodiments, the one or more cultures are further incubated at a temperature below body temperature for a period of about 1 to 7 about days, or about 3-5 days.

In some embodiments, a dual track culturing setup is followed by subjecting a first set of sample portions to a short-track monitoring period and a second set of sample portions to a long-track monitoring period, where the short and long-track monitoring periods have durations according to the above ranges, with the short-track duration being shorter than the long-track duration. Such a dual-track setup has shown good results by enabling faster results from the short-track (e.g., about 1 to about 7 days or about 3 to about 5 days), when possible, without missing the detection of other, slower species and/or strains resulting from the long-track (e.g., about 7 to about 21 days, or about 14 days).

### EXAMPLES

### Example 1

Blood samples were collected from over 600 subjects. More than 30 separate synovial fluid samples and 1 lymphatic fluid sample were also collected. For each sample, about 0.5 ml or less of the sample (about 2 drops) was added to a tube containing 10-15 ml of bovine serum and a tube containing 10-15 ml of BHI broth. The inoculated tubes were incubated at 27° C. Development of L-form culture was monitored by preparing wet mount live slides daily. Samples were monitored for a period of up to 30 days. Samples that showed indications of L-form bacterial growth were typically incubated for at least 48 hours, and typically began to show signs of progressive growth within 48-72 hours. L-form bacteria were not observed to progress to a CWS form while within the broth.

For samples in which L-form bacterial growth was detected, the broth was used to inoculate a variety of agarose plates (mannitol salt, BHI, tryptic soy, tryptic soy w/5% sheep's blood, chocolate blood, Vogel Johnson, Simmons citrate, Columbia, brewer's yeast, nutrient, MacConkey agar, starch agar, and Kligler Iron agar). The inoculant was immediately covered with a sterile cover slide to prevent dehydration of L-form bacteria. Extra inoculant was streaked onto remaining portions of the agarose surface. A set of plates was then incubated at 37° C. in an aerobic incubation unit, and a set of plates was incubated at 37° C. in an anaerobic chamber. Sterile water was supplied in order to maintain a humid environment within the incubation areas. The plates were placed agarose-side down for 12 hours and then were flipped to agarose-side up and incubated for an additional 48 hours. Plates were then removed and sealed in a plastic bag in order to retain moisture and were further incubated at 27° C. for 5 days. At 5 days, plates were inspected for growth and hemolytic activity (on relevant plates). Each colony was transferred (isolated) to a set of nutrient agar and blood agar (trypticase soy agar with 5% sheep blood) plates.

Isolated colonies were characterized using a BioLog GEN III MicroPlate 96 well plate as well as 16S rDNA sequence analysis. The L-form growth protocols have resulted in the culture and isolation of over 254 unique strains of bacteria, including 75 known pathogens, originally residing in respective samples as L-form bacteria.

### Example 2

A comparative study was conducted to compare a standard culturing process to the process of Example 1. Each sample was divided into two portions. The first portion was used to directly inoculate two nutrient agars, which were then incubated and monitored for growth. The second portion was used as inoculant in the L-form growth protocol of Example 1. Results of the comparative study are shown in Table 1 (samples which showed no growth in either protocol are omitted).

TABLE 1

| Sample | Bacteria cultured via direct | Bacteria cultured via process of |
|---|---|---|
| Type | inoculation | Example 1 |
| Blood | No growth | Acintobacter genomospecies 15tu |
| | | Bacillus pumilus/safensis |
| | | Bordetella parapertussis |
| | | Simplicispira metamorpha |
| | | Micrococcus luteus A |
| | | Bacillus salentarsenatis/jeotigaii |
| | | Moraxella canis |
| | | Unknown Rod |
| Blood | Bacillus pumilus/safensis | Bacillus pumilus/safensis |
| | Staph. capitis ss urealyticus | Bacillus pumilus/safensis |
| | | Bacillus pumilus/safensis |
| | | Bacillus thuringiensis/cereus |
| | | Bacilus Vallismortis/subtilis |
| Blood | No growth | Bacillus plakortidis |
| | | Brachybacterium sacelii (26C) |
| | | Unknown Bacteria |
| Blood | No growth | Bacillus pumilus/safensis |
| Blood | No growth | Bacillus pumilus/safensis |
| Blood | No growth | Bacillus lichenformis |
| | | Bacillus lichenformis |
| | | Staphylococcus intermedius |
| Blood | No growth | Bacillus pumilus/safensis |
| Blood | No growth | Bacillus pumilus/safensis |
| | | Micrococcus luteus B |
| | | Corynebacterium terpenotabidum |
| Blood | No growth | Bacillus pumilus/safensis |
| Blood | No growth | Unknown rod |

As shown, growth and culture of L-form bacteria to identifiable classic form was achieved using the process of L-form growth protocol of Example 1, even for many samples which gave no results and no growth under a standard direct inoculation technique. The results show that use of the L-form growth protocol can significantly improve the ability to screen for and then culture and produce L-form capable bacteria.

### Example 3

Eleven cord blood samples (unit 1 through unit 11) were collected and were tested against various cultures of L-form capable bacteria (isolated from blood samples) as well as against a classic-form E. coli strain (isolated from a urine sample). The L-form capable bacteria were all capable of reverting to L-form through hydrostatic manipulation without requiring the use of antibiotic pressure, whereas the E. coli strain was only able to shift to an L-form morphology with the use of heavy antibiotic pressure.

Five of the cord blood samples (units 2, 6, 8, 10, and 11) produced a zone of inhibition when added to solid media plates of the L-form capable bacteria. None of the cord blood samples showed any activity against the classic-form E. coli bacteria.

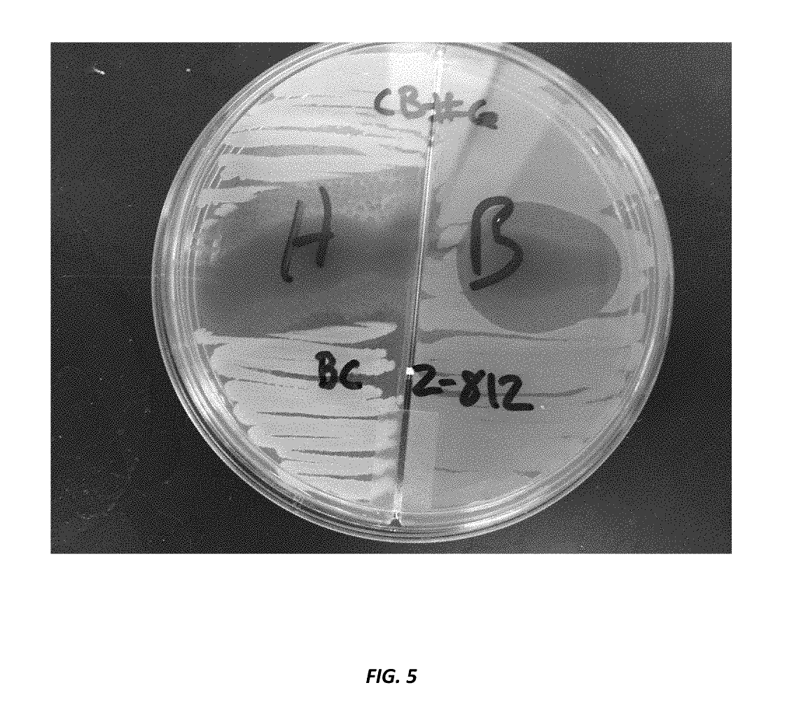

FIG. 5 shows a plate inoculated with L-form capable Bacillus stratosphericus Z-812 (isolated from the blood of a patient having a venous blood malformation). The plate was divided to have LB agar (shown on the left in the photograph) and BHI agar (shown on the right in the photograph). A zone of inhibition of about 4 mm was produced around three drops of cord blood. The photograph is shown at 12 hours post inoculation.

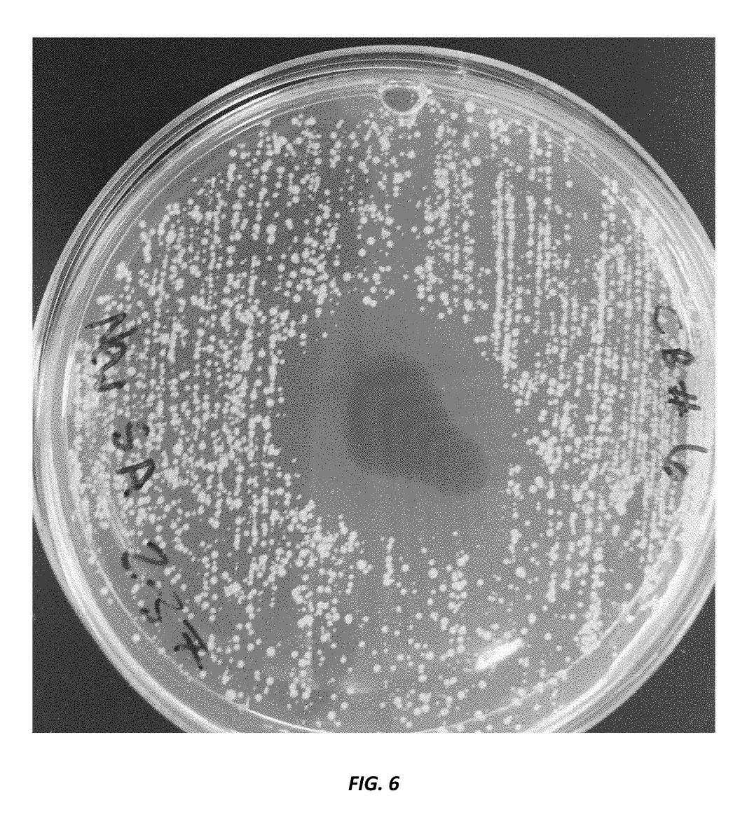

FIG. 6 shows a plate inoculated with L-form capable Staphylococcus aureus (isolated from a patient with Lyme disease) on BHI agar. A zone of inhibition of about 5 mm was produced around three drops of cord blood. The photograph is shown at 12 hours post inoculation.

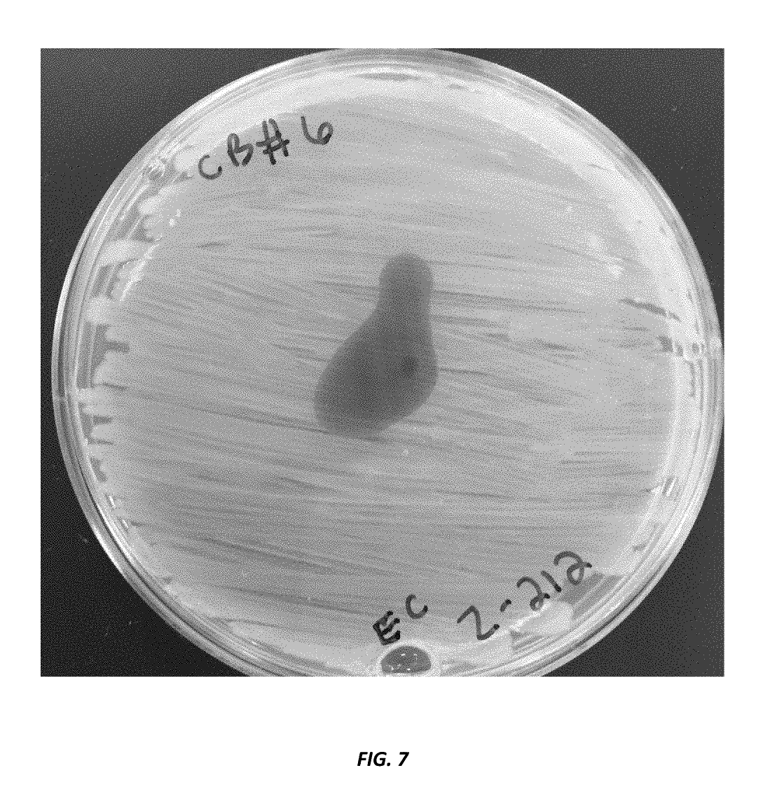

FIG. 7 shows a plate inoculated with classic-form E. coli isolated from a urine sample. As shown, the three drops of cord blood had no inhibitory effect against the classic-form E. coli.

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

1. A method of screening one or more cord blood samples for antibacterial activity against L-form bacteria, the method comprising:
providing an L-form bacteria of interest;
providing one or more cord blood samples, the one or more cord blood samples including whole cord blood and/or one or more cord blood fractions;
contacting the one or more cord blood samples with the L-form bacteria of interest; and
based on the presence or absence of an antibacterial effect against the L-form bacteria of interest, identifying the one or more cord blood samples as effective against L-form bacteria.

2. The method of claim 1, wherein the L-form bacteria of interest is isolated from a subject infected with the L-form bacteria of interest.

3. The method of claim 1 or claim 2, wherein the cord blood sample includes one or more of a plasma, buffy coat, or erythrocyte fraction.

4. The method of claim 1 or claim 2, wherein the cord blood sample includes whole cord blood.

5. The method of any one of claims 1 through 4, wherein the cord blood sample is human cord blood.

6. The method of any one of claims 1 through 5, wherein the L-form bacteria of interest is isolated according to a process comprising:
contacting a biological sample obtained from a subject to a first growth medium, the biological sample being free of bacteria having a cell-wall-sufficient morphology;
incubating the first growth medium under a first set of incubation conditions to promote growth of L-form bacteria present within the sample;
transferring at least a portion of the first growth medium, as an inoculant, to a second growth medium under conditions that maintain a hydrated state of the inoculant; and
incubating the second growth medium under a second set of incubation conditions to promote the progression of the L-form bacteria to a cell-wall-sufficient morphology.

7. The method of any one of claims 1 through 6, the method further comprising treating a subject having an infection of the L-form bacteria of interest with one or more cord blood agents identified as being effective against the L-form bacteria of interest.

8. The method of claim 7, wherein the treatment includes performing a blood transfusion to deliver the one or more cord blood agents.

9. A method of treating a subject having an L-form bacterial infection, the method comprising:
identifying a subject having an L-form bacterial infection;
administering an effective dose of a cord blood agent to the subject; and
the cord blood agent killing or deactivating L-form bacteria causing the L-form bacterial infection.

10. The method of claim 9, wherein the cord blood agent includes whole cord blood.

11. The method of claim 9 or claim 10, wherein the cord blood agent includes one or more of a plasma, buffy coat, or erythrocyte fraction.

12. The method of any one of claims 9 through 11, wherein the L-form bacteria underlying the L-form bacterial infection of the subject is isolated through a process comprising:
contacting a biological sample obtained from a subject to a first growth medium, the biological sample being free of bacteria having a cell-wall-sufficient morphology;
incubating the first growth medium under a first set of incubation conditions to promote growth of L-form bacteria present within the sample;
transferring at least a portion of the first growth medium, as an inoculant, to a second growth medium under conditions that maintain a hydrated state of the inoculant; and
incubating the second growth medium under a second set of incubation conditions to promote the progression of the L-form bacteria to a cell-wall-sufficient morphology.

13. The method of claim 12, wherein a set of cord blood agents are screened against the isolated L-form bacteria, and wherein the cord blood agent administered to the subject is selected from the set of cord blood agents based on effectiveness during the screening.

14. The method of any one of claims 9 through 13, wherein administering an effective dose of a cord blood agent to the subject includes administering a cord blood agent transfusion.

15. The method of any one of claims 9 through 13, wherein the cord blood agent is human cord blood and/or a human cord blood fraction.

16. A method for killing or inhibiting L-form bacteria in an in vitro or ex vivo environment, the method comprising:
providing a culturable form of an L-form bacteria in vitro or ex vivo;
contacting a cord blood agent to the L-form bacteria; and
the cord blood agent killing or deactivating the L-form bacteria.

17. The method of claim 16, wherein the L-form bacteria are provided on solid or liquid media.

18. The method of claim 16, wherein the L-form bacteria are provided within a biological sample.

19. The method of claim 18, wherein the biological sample is a blood sample.

20. The method of any one of claims 16 through 19, wherein the culturable form of the L-form bacteria is provided through a process comprising:
contacting a biological sample obtained from a subject to a first growth medium, the biological sample being free of bacteria having a cell-wall-sufficient morphology;
incubating the first growth medium under a first set of incubation conditions to promote growth of L-form bacteria present within the sample;
transferring at least a portion of the first growth medium, as an inoculant, to a second growth medium under conditions that maintain a hydrated state of the inoculant; and
incubating the second growth medium under a second set of incubation conditions to promote the progression of the L-form bacteria to a cell-wall-sufficient morphology.
